(19) United States
(12) Patent Application Publication
(10) Pub. No.: US 2008/0058590 A1
(43) Pub. Date: Mar. 6, 2008

(54) TISSUE VISUALIZATION DEVICE HAVING MULTI-SEGMENTED FRAME

(75) Inventors: Vahid Saadat, Saratoga, CA (US); Ruey-Feng Peh, Mountain View, CA (US); Edmund A. Tam, Mountain View, CA (US)

Correspondence Address: LEVINE BAGADE HAN LLP, 2483 EAST BAYSHORE ROAD, SUITE 100, PALO ALTO, CA, 94303, US

(73) Assignee: Nidus Medical, LLC., Saratoga, CA (US)

(21) Appl. No.: 11/848,193

(22) Filed: Aug. 30, 2007

Related U.S. Application Data
(60) Provisional application No. 60/824,417, filed on Sep. 1, 2006.

(51) Int. Cl. A61B 1/32

(52) U.S. Cl. 600/109; 606/198

(57) ABSTRACT

Tissue visualization devices having multi-segmented frame are described herein where such devices may utilize multiple expanding frame members coupled to a flexible deployment catheter shaft or rigid shaft. The multiple frame members may extend distally to collapse into a low-profile configuration and may further expand or open radially to create a working field between the frame members. Moreover, the distal ends of each frame member may be tapered such that the frame members may close tightly relative to one another forming an atraumatic end. Additionally, any number of therapeutic tools can also be passed through the catheter or shaft for performing any number of procedures on the tissue for identifying, locating, and/or treating tissue.

### CROSS-REFERENCE TO RELATED APPLICATIONS

This application claims the benefit of priority to U.S. Prov. Pat. App. 60/824,417 filed Sep. 1, 2006, which is incorporated herein by reference in its entirety.

### FIELD OF THE INVENTION

The present invention relates generally to medical devices used for accessing, visualizing, and/or treating regions of tissue within a body. More particularly, the present invention relates to tissue visualization devices having an expandable multi-segmented frame for accessing and/or treating tissue within a patient.

### BACKGROUND OF THE INVENTION

Conventional devices for accessing and visualizing interior regions of a body lumen are known. For example, ultrasound devices have been used to produce images from within a body in vivo. Ultrasound has been used both with and without contrast agents, which typically enhance ultrasound-derived images.

Other conventional methods have utilized catheters, endoscopes, or probes having position sensors deployed within the body lumen, such as the interior of a cardiac chamber, the peritoneal or thoracic cavities, etc. Another conventional device utilizes an inflatable balloon which is typically introduced intravascularly in a deflated state and then inflated against the tissue region to be examined. Imaging is typically accomplished by an optical fiber or other apparatus such as electronic chips for viewing the tissue through the membrane(s) of the inflated balloon. Moreover, the balloon must generally be inflated for imaging. Other conventional balloons utilize a cavity or depression formed at a distal end of the inflated balloon. This cavity or depression is pressed against the tissue to be examined and is flushed with a clear fluid to provide a clear pathway through the blood.

However, such imaging balloons have many inherent disadvantages. For instance, such balloons generally require that the balloon be inflated to a relatively large size which may undesirably displace surrounding tissue and interfere with fine positioning of the imaging system against the tissue. Moreover, the working area created by such inflatable balloons are generally cramped and limited in size. Furthermore, inflated balloons may be susceptible to pressure changes in the surrounding fluid. Additionally, in other body lumens or cavities, the surrounding tissue may collapse or intrude within the environment around the working distal end of the catheter, thus requiring a separate tissue retraction instrument or insufflation of the body cavity, if suitable. However, such additional instruments and insufflation of the body introduces additional complications and time into a procedure.

Accordingly, these types of imaging modalities are generally unable to provide desirable images useful for sufficient diagnosis and therapy of the endoluminal structure. Moreover, anatomic structures within the body can occlude or obstruct the image acquisition process. Also, the presence and movement of opaque bodily fluids such as blood generally make in vivo imaging of tissue regions within the heart difficult.

Other external imaging modalities are also conventionally utilized. For example, computed tomography (CT) and magnetic resonance imaging (MRI) are typical modalities which are widely used to obtain images of body lumens. However, such imaging modalities fail to provide real-time imaging for intra-operative therapeutic procedures. Fluoroscopic imaging, for instance, is widely used to identify anatomic landmarks within the heart and other regions of the body. However, fluoroscopy fails to provide an accurate image of the tissue quality or surface and also fails to provide for instrumentation for performing tissue manipulation or other therapeutic procedures upon the visualized tissue regions. In addition, fluoroscopy provides a shadow of the intervening tissue onto a plate or sensor when it may be desirable to view the intraluminal surface of the tissue to diagnose pathologies or to perform some form of therapy on it.

Moreover, many of the conventional imaging systems lack the capability to provide therapeutic treatments or are difficult to manipulate in providing effective therapies. Thus, a tissue imaging system which is able to provide real-time in vivo access to and images of tissue regions within body lumens and which also provide instruments for therapeutic procedures upon the visualized tissue are desirable.

### SUMMARY OF THE INVENTION

An instrument having a low-profile configuration for delivery into and/or through a body and an expandable assembly may be used for retracting or moving tissue from a working distal end of the assembly by utilizing an expandable frame to create a working theater within the body without the need for additional instrumentation. Such an apparatus provides a platform for minimally invasive visualization and therapeutics treatment to be carried out for a variety of procedures in different areas including, but not limited to, e.g., trans-septal access and/or patent foramen ovale closure in cardiac surgery, cutting of the corrugator muscle and accessing the breast from the navel in cosmetic surgery, placing of neuro-stimulator lead for pain management, implanting of artificial disks and injecting of artificial nucleus to the spine, visualization and treatment of the heart/lungs with a sub-xiphoid approach in percutaneous surgery, etc.

One variation of such an instrument assembly may have several segmented frame members extending distally from a deployment catheter. These frame members may collapse into a low-profile configuration where the distal ends of each frame member may be tapered such that the frame members close tightly relative to one another to form an atraumatic or blunted end. The frame may be held in a closed configuration without the aid of a sheath although other variations may utilize a slidable outer sheath to slide over and collapse and/or expand the multi-segmented frame. Each frame member may comprise a rigid body that can be made from any number of materials, e.g., Titanium, stainless steel, or hard plastics such as thermoset plastics, polycarbonate, polyurethane, polysulfone, or other thermoset materials, etc.

One or more lumens may be defined through the catheter and the distal ends of the frame members may collectively form an opening to accommodate the passage of an instrument or guidewire therethrough to facilitate guidance and/or delivery within the patient body, particularly for intravascular advancement or introduction through an opening in tissue. The atraumatic or blunted end of the frame members may form a tapered profile such that the distal end of the collapsed frame members may be utilized optionally as a dilator for introduction into and/or through tissue openings.

Once the assembly has been introduced into the body cavity or advanced through the patient vasculature and is desirably positioned for visualization and/or treatment upon an underlying tissue region, the individual frame members may be opened radially relative to the catheter to form a conically-shaped hood. Each of the segments may be articulated to radially reconfigure at an angle relative to a longitudinal axis defined by the elongated catheter. The gaps in-between the deployed frame members may have a distensible or reconfigurable flexible membrane, such as a foldable plastic or latex flaps, extending beneath and/or between the frame members. These flaps may be folded, collapsed, or otherwise hidden within the frame when the device is in the closed position. Upon expansion or opening of the frame members, the membrane may distend or unfold between each adjacent frame member to form an open area defined within the frame members and flaps which is open distally to the environment. As frame members radially extend, one or more openings within the distal end of catheter may be exposed.

The deployment or retraction of the frame members relative to the catheter can be controlled by any number of mechanisms such as pullwires, hydraulics, electric motor-driven gears, cams, or linkages, etc. These mechanisms may be embedded within the elongated catheter and coupled to one or more frame members to control the opening and/or closing.

The catheter shaft may be configured to be flexible; however, other variations may include a rigid shaft such that the assembly may be utilized much like a laparoscopic instrument. Additionally, imaging elements such as optical fiberscopes, CMOS or CCD cameras, etc. may be positioned within the open area or off-axis relative to a longitudinal axis of the catheter and/or frame members.

### BRIEF DESCRIPTION OF THE DRAWINGS

FIGS. 1A and 1B show one variation of a tissue expansion and visualization assembly in a closed low-profile and partially-opened configuration.

FIG. 1C shows the assembly of FIG. 1A in its fully expanded configuration.

FIG. 2 shows a side view of the tissue expansion and visualization assembly disposed upon a catheter advanced through an outer sheath.

FIG. 3 shows another variation of the assembly having an expandable transparent balloon positioned therewithin to facilitate visualization.

FIG. 4 shows another variation of the assembly having a rigid shaft and an imaging assembly, e.g. CMOS, CCD, or fiberscope, and helical tissue engager extending from a working channel.

FIGS. 5A and 5B show another variation of the assembly deployed from a Verres-type needle sheath.

FIGS. 6A and 6B show yet another variation of the device having a Verres-type needle advanced through the closed segmented frame members.

FIGS. 7A to 7C show yet another version of the assembly having a guidewire rapid-exchange feature.

FIGS. 8A and 8B show side and perspective views, respectively, of another variation of the assembly having an imaging element positioned longitudinally relative to the closed segmented frame members.

FIGS. 9A and 9B show side and perspective views, respectively, of the assembly having the segmented frame members and barrier expanded with the imaging element positioned distally of the hood.

FIGS. 10A and 10B show side and perspective views, respectively, of the assembly having the camera pulled proximally into the off-axis channel or pocket clearing the open area within the expanded assembly for advancement of an instrument therethrough for performing a procedure upon the underlying tissue.

### DETAILED DESCRIPTION OF THE INVENTION

In performing any number of procedures within a body lumen or body cavity, such as within a heart chamber, peritoneal or thoracic cavity, etc. of a patient, an instrument having a low-profile configuration for delivery into and/or through a body and an expandable assembly for retracting or moving tissue from a working distal end of the assembly may utilize an expandable frame to create a working theater within the body without the need for additional instrumentation. Such an apparatus provides a platform for minimally invasive visualization and therapeutics treatment to be carried out for a variety of procedures in different areas including, but not limited to, e.g., trans-septal access and/or patent foramen ovale closure in cardiac surgery, cutting of the corrugator muscle and accessing the breast from the navel in cosmetic surgery, placing of neuro-stimulator lead for pain management, implanting of artificial disks and injecting of artificial nucleus to the spine, visualization and treatment of the heart/lungs with a sub-xiphoid approach in percutaneous surgery, etc.

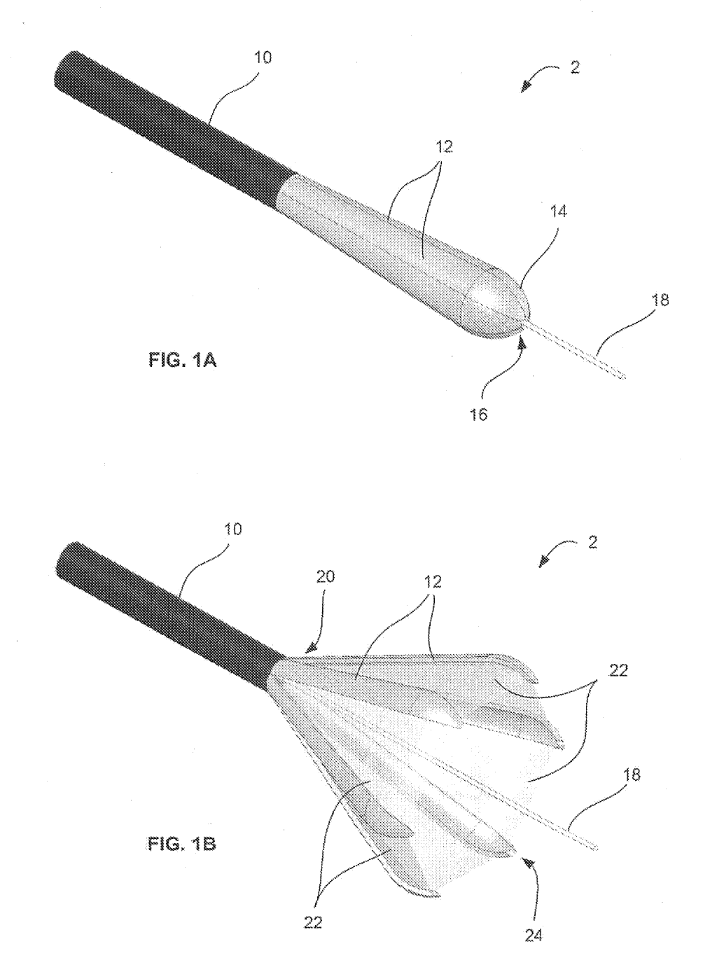

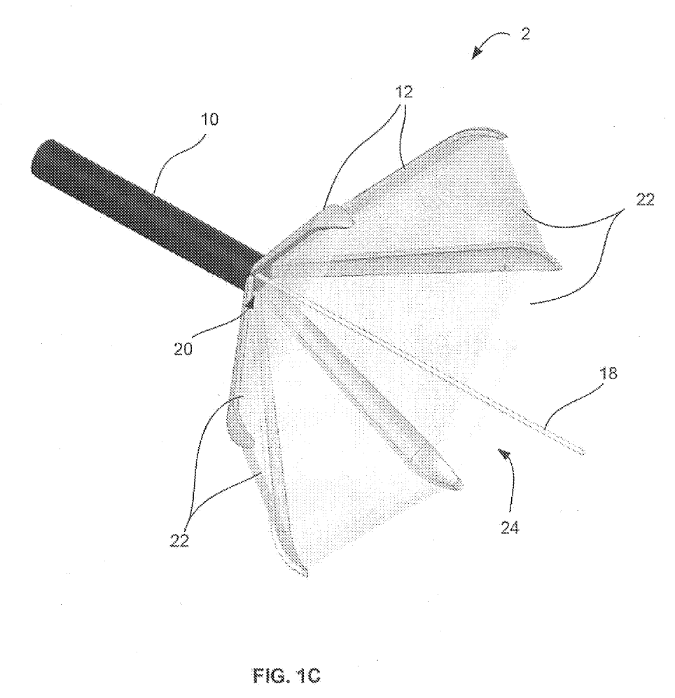

Turning now to FIG. 1A, a tissue visualization and treatment assembly 2 is illustrated in perspective view having several segmented frame members 12 extending distally from deployment catheter 10 which collapse into a low-profile configuration where the distal ends of each frame member 12 may be tapered such that the frame members may close tightly relative to one another forming an atraumatic or blunted end 14. The frame 12 may be held in a closed configuration without the aid of a sheath although other variations may utilize a slidable outer sheath to slide over and collapse and/or expand the multi-segmented frame. Each frame member 12 may comprise a rigid body that can be made from any number of materials, e.g., Titanium, stainless steel, or hard plastics such as thermoset plastics, polycarbonate, polyurethane, polysulfone, or other thermoset materials, etc.

One or more lumens may be defined through the catheter 10 and the distal ends of the frame members 12 may collectively form an opening 16 to accommodate the passage of an instrument or guidewire 18 therethrough to facilitate guidance and/or delivery within the patient body, particularly for intravascular advancement or introduction through an opening in tissue. Alternatively, in the absence of a guidewire 18, the distal tips of each frame member 12 may be configured to fit tightly against one another without defining such an opening. A hydrophilic coating may be optionally applied on the frame members 12 and the guidewire 18 to create a low friction interface between the frame members and the guidewire 18. The frame members 12 may be appropriately sized such that when the hood is in the closed configuration, adequate clearance is provided to allow the guidewire 18 to slide freely between the frame members 12. The atraumatic or blunted end 14 of the frame members 12 may form a tapered profile such that the distal end of the collapsed frame members 12 may be utilized optionally as a dilator for introduction into and/or through tissue openings.

Once assembly 2 has been introduced into the body cavity or advanced through the patient vasculature and is desirably positioned for visualization and/or treatment upon an underlying tissue region, the individual frame members 12 may be opened radially relative to catheter 10 to form a conically-shaped hood, as shown in the partially-opened configuration of FIG. 1B. Although the variation illustrates six frame members 12 radially positioned in a uniform configuration about the distal end of the flexible catheter 10, fewer than six or more than six frame members 12 may be utilized depending upon the desired configuration in alternative variations. Moreover, each of the frame members 12 may be irregularly positioned relative to one another so long as the frame members 12 may collapse into a low-profile shape.

Each of the segments 12 may be articulated to radially reconfigure at an angle relative to a longitudinal axis defined by the elongated catheter 10. The gaps in-between the deployed frame members 12 may have a distensible or reconfigurable flexible membrane 22, such as a foldable plastic or latex flaps, extending beneath and/or between the frame members 12. These flaps 22 may be: folded, collapsed, or otherwise hidden within the frame 12 when the device is in the closed position. Upon expansion or opening of the frame members 12, the membrane 22 may distend or unfold between each adjacent frame member 12 to form an open area 24 defined within the frame members 12 and flaps 22 which is open distally to the environment. As frame members 12 radially extend, one or more openings 20 within the distal end of catheter 10 may be exposed.

FIG. 1C shows the assembly in its fully expanded configuration. The deployment or retraction of the frame members 12 relative to the catheter 10 can be controlled by any number of mechanisms such as pullwires, hydraulics, electric motor-driven gears, cams, or linkages, etc. These mechanisms may be embedded within the elongated catheter 10 and coupled to one or more frame members 12 to control the opening and/or closing. Accordingly, adequate force transmission may be generated as the articulation motion may be utilized to enlarge or retract tissue bodies, open or expand body lumens, open tissue flaps or dissect obstructions found within body lumens, among other uses. Moreover, frame members 12 may be extended into various angles relative to catheter 10 to widen or narrow the open area 24 depending upon the tissue region and anatomy to be visualized and/or treated.

As previously mentioned, the device may define multiple lumens or channels therethrough which may be utilized for any number of instruments, such as an optical channel where optical fibers are positionable for providing direct visualization, an irrigation channel for fluid injection (e.g., saline can be injected to flush away opaque fluids or any obstructing debris within the space 24 created by the frame), etc. The multi-lumen channel may also include working channels in which tools or instruments such as guidewires, needles, biopsy forceps, scissors, helical tissue engagers, electrode sensors or ablation probes, etc. can be inserted. Details of utilizing the expanded frame as a hood for displacing blood therewithin with a transparent fluid for visualization through the fluid of the underlying tissue surrounded by the frame are shown and described in further detail in U.S. Pat. Pub. 2006/0184048A1 and 2007/0167828A1, which are each incorporated herein by reference in their entirety.

As previously mentioned, the multi-segment frame 12 in its closed configuration may form a blunt and/or rounded atraumatic distal end 14. This configuration may be used for navigation and/or burrowing through tissue lumens such as arteries, blood vessels, chambers of the heart, subcutaneously within areas underneath the skin, gastrointestinal tract or the respiratory tract, etc. In the closed configuration, the blunt and smooth distal end 14 may enable the assembly to burrow along body lumens smoothly. Torquing action about the longitudinal axis may also be utilized to further facilitate such threading and navigating motions. The frame members 12, when constructed by transparent materials such as fiberglass, may enable an imaging element positioned within the frame members 12 to visualize the surrounding tissue directly through the frame members 12 during navigation and/or burrowing through tissue.

The assembly may also be utilized to penetrate and/or navigate directly through tissue. This can be achieved by penetrating a needle through a target tissue from the working channel of the device. Guidewire 18 may be disposed at the penetration spot within the tissue while the needle is removed. The multi-segment frame 12 can then be closed, as shown in FIG. 1A, with the guidewire 18 still in place protruding from the closed frame. The device may then track along guidewire 18 and navigate through the penetrated tissue to access to the distal side of the tissue.

The frame members 12 may be opened whenever visualization through the open area is desired. The frame members 12 can also be opened when a tissue lumen is to be enlarged or tissue bodies require retraction or repositioning. The frame members 12 can also be opened when one or more tools are to be deployed to treat a target tissue area. The open area 24 formed by opened frame members 12 provides a therapeutic theater or area for the user to conduct therapeutic treatments under direct visualization.

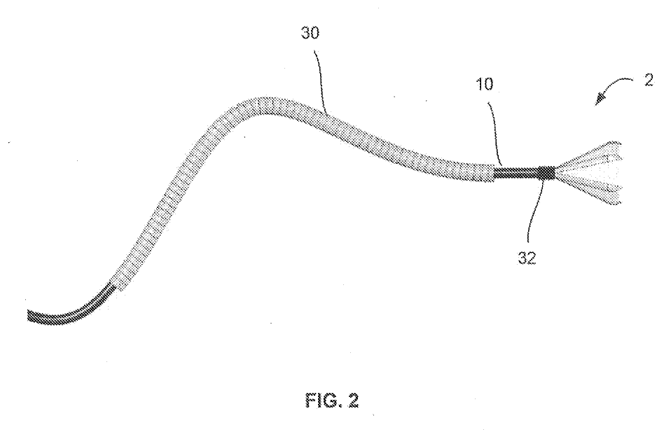

FIG. 2 shows a side view of an endoscopic or flexible version of the tissue visualization assembly with the expandable multi-segmented frame 12. Accompanying the flexible elongated catheter 10 may be an outer sheath 30 which may facilitate closing collapse of the frame members 12 between their open and closed configurations by respectively retracting or advancing sheath 30 relative to catheter 10. Additionally and/or optionally, catheter 10 or sheath 30 may incorporate an articulatable neck portion 32. Articulation of portion 32 may enable navigation of the assembly to allow steering as the assembly is advanced in or through a body lumen. The articulation and navigation may be controlled precisely by incorporating a catheter under robotic control technology developed by Hansen Medical, Inc. (Sunnyvale, Calif.). Additionally and/or alternatively, the articulation and navigation can also be controlled precisely utilizing, for instance, a controllable magnetic field utilizing technology developed by Stereotaxis, Inc (Saint Louis, Mo.). In such an alternative, the frame members may be fabricated of ferrous magnetic materials directly or they may incorporate a ferrous magnet attached or integrated along the device and/or frame members 12. Examples of such technologies which may be utilized with the assembly described herein are shown and described in further detail in U.S. Prov. Pat. App. 60/824,421 filed Sep. 1, 2006 and U.S. patent application Ser. No. 11/______, filed Aug. ______, 2007 (Attorney Docket No. VYMD-N-Z010.00-US), each of which are incorporated herein by reference in their entirety.

In certain procedures such as for cardiac surgery, the assembly 2 may be utilized for catheter-based treatments of indications such as structural heart diseases or chronic total occlusion applications, amongst others. The multi-segment frame 12 can be advanced intravascularly into the chambers of the heart, for instance, via the inferior or superior vena cava and into the right atrium. The assembly may also be utilized to obtain trans-septal access to the left atrium to perform treatments such as atrial fibrillation ablation, mitral valvuloplasty, left atrial appendage closure or patent foramen ovale closure, among other procedures. Additionally, the device may also be utilized to advance through vessels such as arteries to clear plaques that may be obstructing blood flow while under direct visualization.

The device is also applicable in cosmetics surgeries for procedures such as cutting of the corrugator muscle in the forehead by navigating subcutaneously under the skin to access to the forehead of the patient minimizing damage to the surrounding tissues, unlike conventional procedures or tools. Similarly, the assembly 2 can be advanced percutaneously through the navel of the patient such that the assembly 2 can access the breast of the patient to perform diagnostics or cosmetic treatment to this area. The assembly 2 may also be able to be advanced subcutaneously under the skin or through narrow lumens of the body for applications in pain management therapies, for instance, by navigating and placing one or more neuro-stimulator leads at the target nerve site for pain management control.

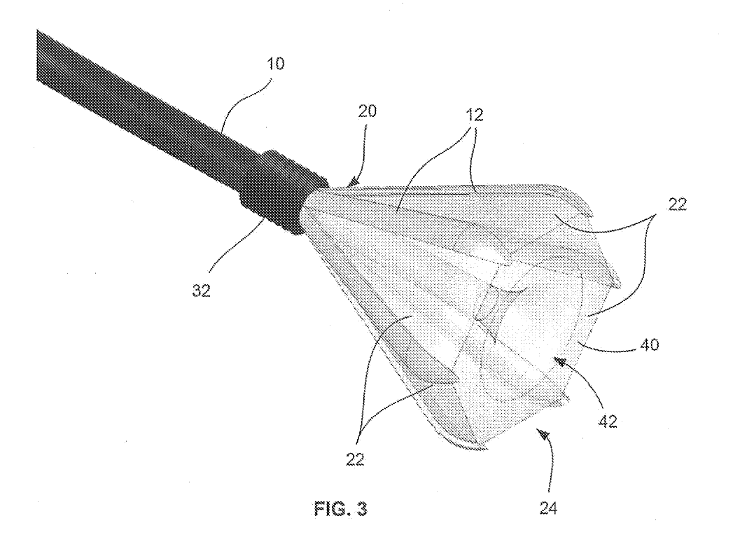

FIG. 3 shows a perspective view of another variation of the assembly having an optional circumferential balloon 40 inflatable within the open area 24. Balloon 40 may define a channel 42 through the center portion of balloon 40 to allow for various instruments to be passed therethrough. The balloon 40 may be expandable from one of the channels and may be fabricated from a transparent material such that visualization through the balloon 40 is possible. The presence of a transparent balloon 40 may be particularly useful in enabling visualization when the device is used in environments where it is submerged in opaque body fluids such as blood. The distal end of the inflated balloon 40, upon contact with a tissue surface of interest, may be able to visualize the tissue surface through the transparent balloon 40 without any obstructions.

Another alternative balloon architecture may include an inflatable balloon attached to the distal end of the elongated shaft and having a working channel defined through the balloon member. The assembly can be housed within the balloon working channel with transparent multi-segmented frames 12 in the closed configuration. Hence, when the balloon is inflated, the device is able to visualize an area much further than the distal end of the frames 12. Another balloon architecture includes having a tubing protruding from the closed frame and a balloon inflated from this tubing. Optical fiberscopes can also be protruded from the closed frame to enable unobstructed visualization through the inflated balloon.

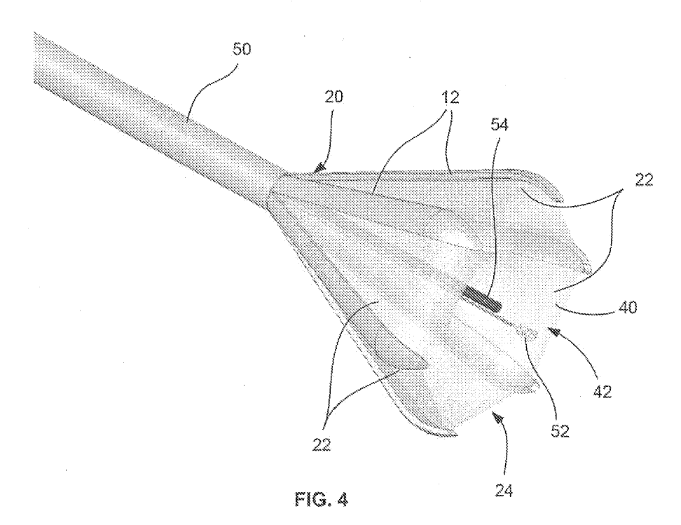

FIG. 4 shows a perspective view of a laparoscopic or rigid version of the assembly having its expandable frame members 12 attached to a rigid elongate shaft 50, which may be made from a rigid shaft to facilitate percutaneous access through an incision made in the patient's skin much like a laparoscopic instrument. Moreover, rigid shaft 50 may provide a stable platform for therapeutic applications when minimally invasively inserted into the body. This particular variation is illustrated as having a helical tissue engagement instrument 52 for temporarily engaging and manipulating a tissue structure. Moreover, an optical fiberscope 54 is illustrated as introduced through shaft 50 and into the open area 24 for providing visualization of the tissue region. However, other imaging assemblies such as CMOS or CCD imagers may be utilized in other variations. The assembly can also be inserted percutaneously to view or treat the exterior of organs such as the stomach, liver, intestines, etc. The assembly is also applicable in percutaneous surgeries such as accessing the exterior of various tissue structures such as the heart or lung utilizing, e.g., a sub-xiphoid approach. Additionally, the assembly can also be minimally invasively inserted into the spine for implanting of devices such as artificial disks, injecting of artificial nucleus or to perform other related spinal treatment, etc.

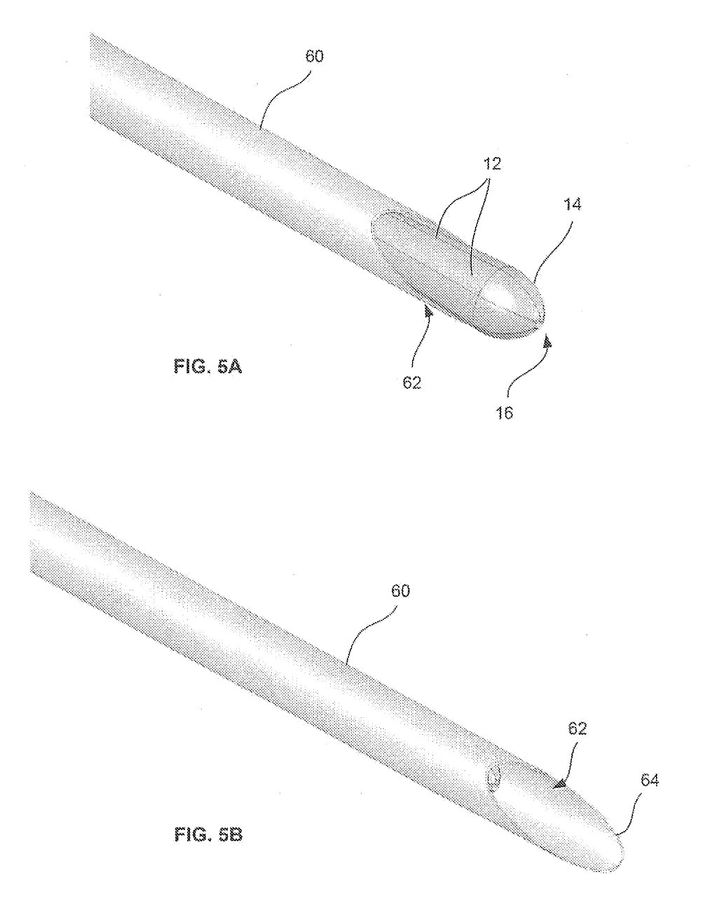

In yet another variation, FIGS. 5A and 5B illustrate perspective views of an assembly utilizing a Verres-type needle feature. As shown in FIG. 5A, a Verres-type needle 60 may positioned around the frame members 12 as an outer sheath with the assembly in their closed configuration functioning as the blunt tip of a Verres needle. During procedures where the frame members 12 are to be pierced into or through a tissue region, initial pressure applied on the assembly may cause the assembly to retract into the lumen 62 of needle 60, as shown in FIG. 5B. Once the tapered needle tip 64 of needle 60 has pierced through the tissue region, frame members 12 may be advanced distally at least partially until the atraumatic distal end 14 extends beyond the piercing tip 64 to function as an atraumatic end. When appropriate, frame members 12 may be further advanced distally relative to needle tip 64 such that frame members 12 may be deployed into their expanded configuration, as described above.

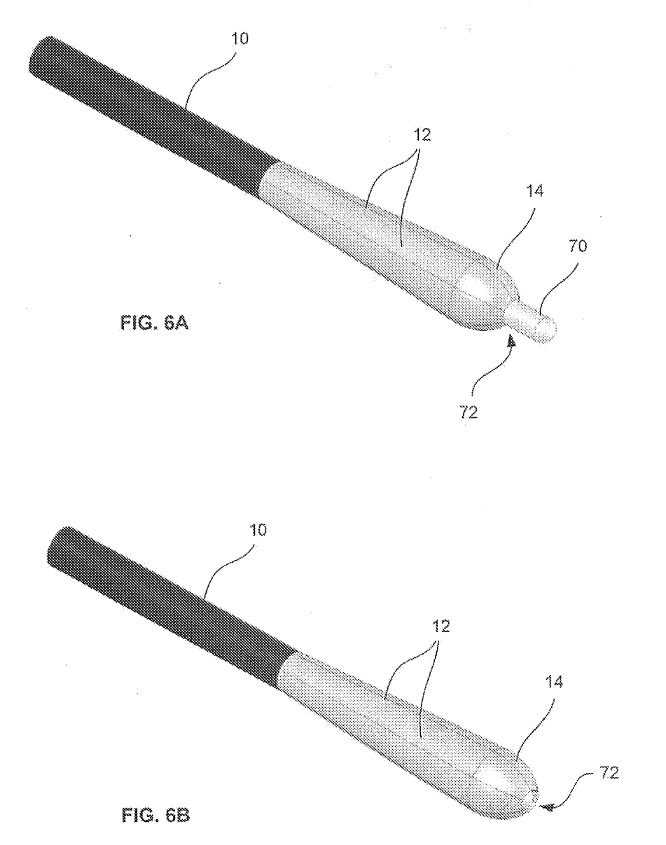

FIGS. 6A and 6B show perspective views of another variation utilizing a Verres-type needle feature. In this variation, the frame members 12 in their closed configuration may incorporate a shaft having a blunt tip 70 positioned through frame members 12 protruding at least partially through opening 72 formed by the closed frame members 12. With blunt tip 70 protruding, the assembly may be advanced into or through tissue, like a boring instrument or dilator. Similar to a Verres needle, the blunt tip 70 may be retracted proximally within the frame members 12 when an initial axial force is applied, as shown in FIG. 6B, and another instrument, such as a needle, may be advanced through opening 72, if so desired. Alternatively, frame members 12 may then be expanded to retract any surrounding tissue and/or to provide visualization of the tissue region adjacent to the open area 24. Both versions of the Verres-type needle feature may function as a safety mechanism for the assembly to prevent or inhibit any inadvertent tissue damage or penetration from occurring. This may be particularly useful in procedures where intensive and/or aggressive burrowing (such as under the skin or through obstructed arteries) is required during a procedure.

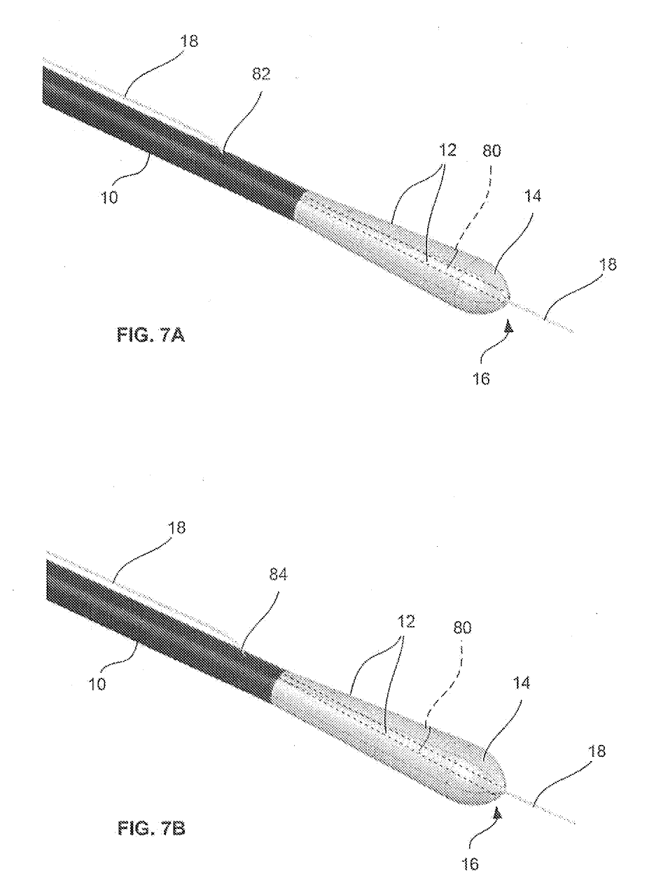

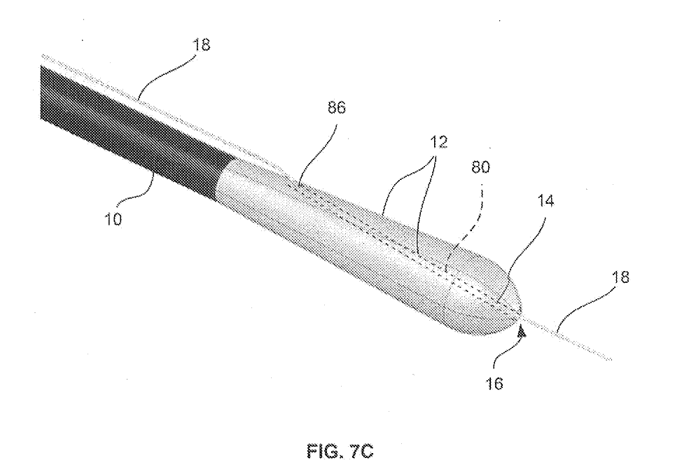

FIGS. 7A to 7C show perspective views of another variation of the tissue visualization catheter having a rapid-exchange feature for exchanging guidewires through the device. A variety of entry points for the guidewire 18 can be seen from the illustrations shown. Each entry point defines a channel or lumen 80 that runs the guidewire 18 from the entry point to the distal end of the frame where the guidewire exits from the tip through opening 16. As illustrated in FIG. 7A, guidewire entry 82 is shown in this variation located at a position which is proximal of frame members 12 along the shaft of catheter 10. FIG. 7B illustrates another variation where guidewire entry 84 is located along catheter 10 just proximal to frame members 12 while FIG. 7C illustrates yet another variation where guidewire entry 86 is located at a position along the frame members 12 rather than catheter 10.

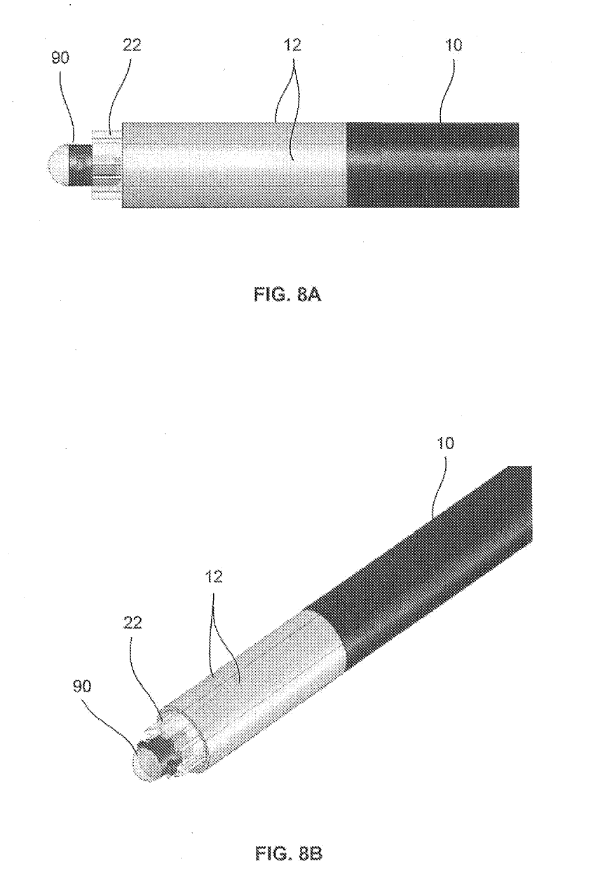

In addition to utilizing an imaging element, such as an optical fiberscope through a working lumen of the catheter 10, other variations of the assembly may utilize imaging elements positioned off-axis relative to a longitudinal axis of catheter 10. With the imaging element positioned off-axis with respect to the catheter 10, the user may gain a relatively larger field of visualization during therapeutic or diagnostic procedures. Imaging element 90, as described above, may comprise an optical fiberscope or a CMOS or CCD imaging camera. FIGS. 8A and 8B show side and perspective views, respectively, of a variation of the assembly where an imaging element 90 may be hidden within or positioned distally of the collapsed frame members 12.

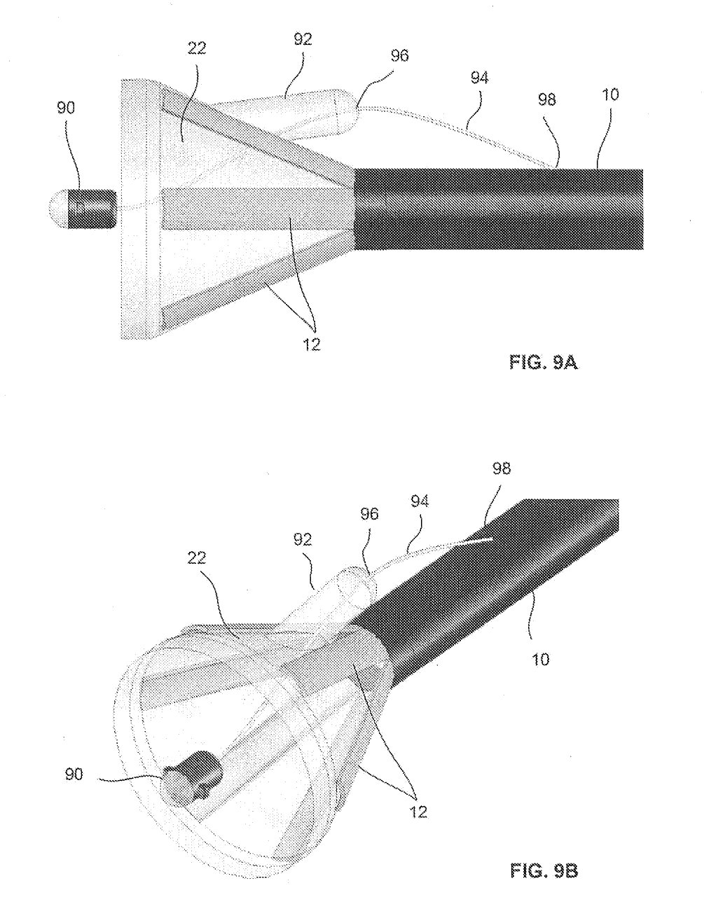

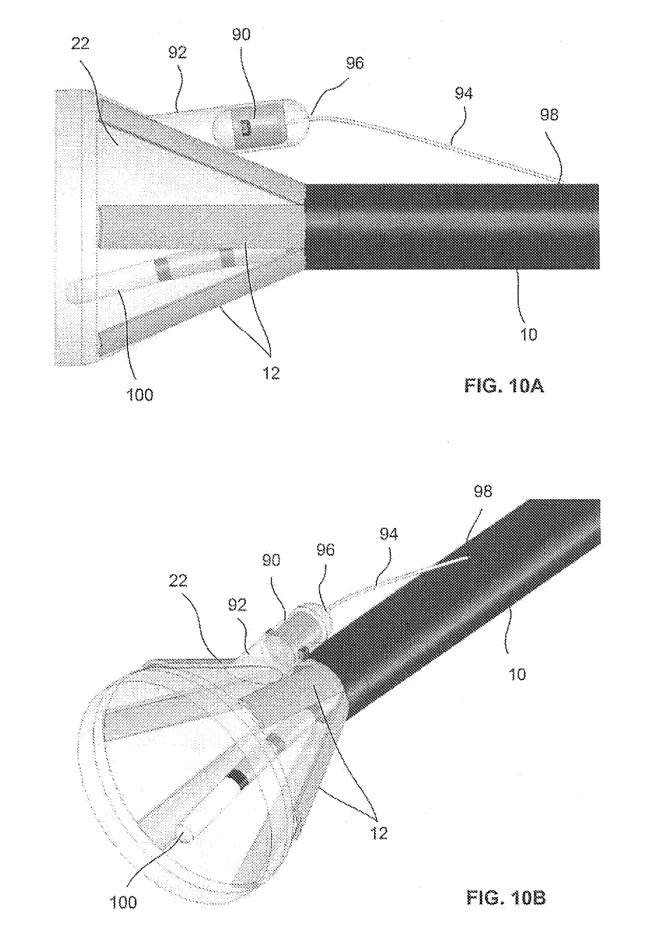

With frame members 12 expanded, as shown in the side and perspective views of FIGS. 9A and 9B, respectively, imaging element 90 may be seen positioned within or distally of frame members 12 while attached to a support member or wire 94 which extends from imaging element 90, through an expandable pocket or receiving channel 92 integrated within membrane 22 between frame members 12, through opening 96 and into or along catheter 10 through opening 98. By pulling support member 94 proximally, imaging element 90 is pulled proximally through frame members 12 and into receiving channel 92, where it may be angled such that imaging element 90 is able to view the underlying tissue region contained within membrane 22 and frame members 12, as shown in the side and perspective views of FIGS. 10A and 10B.

The imaging element 90 may be positioned distally of the collapsed hood by extending the support member 94 distally to facilitate reduction of the catheter profile while maximizing an outer diameter of the catheter 10 to allow relatively larger and/or more economical and/or more powerful imaging elements 90, such as CMOS or CCD cameras, to be utilized.

With imaging element 90 positioned off-axis, various instruments 100 such as RF ablation probes, graspers, needles, etc., can be deployed forward into the open area after imaging element 90 is moved with respect to the frame members 12. Upon further urging of the support member 94, the channel or pocket 92 may also be articulated as the pocket, which may be fabricated from a soft compliable material similar to or the same as membrane 22, may be able to stretch or deform laterally to enable additional movement of imaging element 90 therewithin.

Further examples and details of off-axis configurations for utilizing imaging elements and methods of deploying and/or using such imaging elements are shown and described in further detail in U.S. Prov. Pat. App. 60/871,424 filed Dec. 21, 2006, which is incorporated herein by reference in its entirety.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

1. An apparatus for forming a working area within or upon a tissue region, comprising:
a tubular shaft having a length;
a plurality of frame members extending distally from the tubular shaft and having a low-profile collapsed configuration where the frame members close relative to one another and an expanded configuration where the frame members open radially relative to the tubular shaft such that an open area is formed between the frame members; and
a membrane extending beneath or between each frame member.

2. The apparatus of claim 1 wherein the tubular shaft comprises a flexible catheter having at least one lumen defined therethrough and in communication with the open area.

3. The apparatus of claim 1 wherein the tubular shaft comprises a rigid shaft having an elongate length.

4. The apparatus of claim 3 wherein a distal end of the rigid shaft defines a piercing tip.

5. The apparatus of claim 1 wherein the plurality of frame members form an atraumatic or blunted end when in their collapsed configuration.

6. The apparatus of claim 1 further comprising an outer sheath to slide over the plurality of frame members.

7. The apparatus of claim 1 wherein the frame members comprise a rigid body comprised of a material selected from the group consisting of Titanium, stainless steel, thermoset plastics, polycarbonate, polyurethane, polysulfone, and transparent fiberglass.

8. The apparatus of claim 1 further comprising an instrument or guidewire extending through the tubular shaft and passing at least partially through the open area.

9. The apparatus of claim 8 wherein the plurality of frame members in their collapsed configuration are sized to provide clearance of the instrument or guidewire to slide freely between the frame members.

10. The apparatus of claim 1 further comprising at least one pullwire to articulate the frame members between the collapsed and open configuration.

11. The apparatus of claim 1 further comprising at least one fluid lumen defined through the tubular shaft in communication with the open area.

12. The apparatus of claim 1 further comprising an imaging element positioned within the tubular shaft or along the frame members.

13. The apparatus of claim 12 wherein the imaging element comprises an optical fiberscope, CMOS, or CCD camera.

14. The apparatus of claim 1 wherein the open area is enclosed by the frame members, membrane, and tissue region.

15. The apparatus of claim 1 further comprising an inflatable balloon positioned within the open area.

16. The apparatus of claim 1 wherein the tubular shaft or a frame member defines an opening for passage of a guidewire through the apparatus.

17. A method for treating a tissue region, comprising:
introducing into the tissue region a tubular shaft having a plurality of frame members extending distally from the tubular shaft in a collapsed configuration where the frame members are closed relative to one another;
expanding the frame members open radially relative to the tubular shaft such that an open area is formed within the tissue between the frame members and the tissue region to be treated;
visualizing the tissue region to be treated within the open area.

18. The method of claim 17 wherein introducing comprises advancing the tubular shaft percutaneously or intravascularly into a body lumen or cavity.

19. The method of claim 17 wherein introducing comprises advancing the tubular shaft along a guidewire passing through the frame members.

20. The method of claim 17 wherein expanding comprises retracting the tissue via the frame members.

21. The method of claim 17 wherein expanding further comprises infusing a transparent fluid into the open area such that an opaque fluid within the tissue region is displaced from the open area.

22. The method of claim 21 wherein visualizing comprises viewing the tissue region to be treated through transparent fluid.

23. The method of claim 17 wherein visualizing comprises viewing the tissue region to be treated via an optical fiberscope, CMOS or CCD camera, disposed within or adjacent to the open area.

24. The method of claim 17 further comprising treating the tissue region within the open area via an instrument introduced through the tubular shaft and into the open area.

25. The method of claim 24 wherein treating comprises ablating the tissue region via an energized probe.
